(19) United States
(12) Patent Application Publication
(10) Pub. No.: US 2018/0360368 A1
(43) Pub. Date: Dec. 20, 2018

(54) Method and System for the Assessment and Rehabilitation of Neurologic Deficits

(71) Applicants: Bennett Gatto, Stamping Ground, KY (US); Monte Shaffer, Nicholasville, KY (US)

(72) Inventors: Bennett Gatto, Stamping Ground, KY (US); Monte Shaffer, Nicholasville, KY (US)

(21) Appl. No.: 16/036,924

(22) Filed: Jul. 16, 2018

Related U.S. Application Data
(60) Provisional application No. 62/522,118, filed on Jun. 20, 2017.

(51) Int. Cl. A61B 5/00; A61B 5/11; A61B 5/16; G09B 19/00; A61B 5/05; A61B 5/0205; A61B 5/145; A61B 5/026; A61B 5/0476

(52) U.S. Cl. CPC A61B 5/4082; A61B 5/024; A61B 5/162; A61B 5/4011; A61B 5/4017; G09B 19/00; A61B 5/0022; A61B 5/05; A61B 5/02055; A61B 5/14542; A61B 5/026; A61B 5/14532; A61B 5/163; A61B 5/0476; A61B 5/4803; A61B 5/6897; A61B 5/112; A61B 5/6892; A61B 5/6826; A61B 5/1125; A61B 2562/0219; A61B 5/1124

(57) ABSTRACT

A system and method for the assessment and therapeutic treatment of neurologic function deficits through the evaluation of voluntary and involuntary neuromuscular activity and voluntary neuromuscular responses made on demand as a response to a cognitive skills test. The system permits the remote assessment and therapy of a patient through prescribed physical and cognitive skills regimens either through the remote direction of a therapist or through an artificial intelligence system that selects and modifies regimens based upon the analysis of historical data and/or real-time data. The combination of physical and cognitive therapy to require physical responses to cognitive queries is believed to improve patient outcomes by slowing, halting, or reversing the progression of certain neurological deficits.

### CROSS REFERENCE TO RELATED APPLICATIONS

The present application is related to and derives priority from U.S. Provisional Application 62/522,118 filed on Jul. 16, 2017.

### TECHNICAL FIELD

The presently disclosed subject matter relates generally to cognitive and physical therapy systems and methods to assess and treat neurodegenerative disorders and brain injuries, and more particularly a therapy system to inhibit the progression of Parkinson's Disease through a multi-faceted stimulus and response methodology.

### BACKGROUND

Neurodegenerative disorders and brain injuries are primarily diagnosed through an assessment of physical, sensory, and cognitive functionality. Neurodegenerative disorders especially puzzling and problematic as they are incurable and debilitating conditions that result in progressive degeneration and/or death of nerve cells, primarily in the brain, often affecting physical processes, e.g. movement and breathing, as well as mental processes. Many of these diseases are genetic but some have non-genetic origins, e.g. alcoholism, a tumor, or a stroke. Other causes may include toxins, chemicals, and viruses. Neurodegenerative disorders include Parkinson's disease, Alzheimer's disease, Amyotrophic lateral sclerosis, Friedreich's ataxia, Huntington's disease, and Lewy body disease. There are also auto-immune diseases that affect nerve cells such as Guillain-Barré and multiple sclerosis, although there is increasing evidence that multiple sclerosis is actually a neurodegenerative disorder, rather than an auto-immune response.

Parkinson's disease, i.e. PD, is one of the most well-known neurodegenerative disorders. PD affects predominately dopamine-producing (“dopaminergic”) neurons in the midbrain in the substantia nigra region. Most of the dopamine neurons of the brain originate in the midbrain and are found in either the substantia nigra or the ventral tegmental areas. The ventral tegmental area is located adjacent to the substantia nigra. The substantia nigra is made up of two anatomically and functionally distinct portions: the substantia nigra pars compacta and the substantia nigra pars reticulata. Dopamine neurons are found predominantly in the substantia nigra pars compacta and the death of these neurons is associated with PD, while the pars reticulata is populated largely by gamma-aminobutyric acid, i.e. GABA, neurons. Many of the dopamine neurons of the substantia nigra project to the striatum, another part of the basal ganglia that is made up of the caudate and putamen. In doing so they form a pathway called the nigrostriatal dopamine pathway that is thought to be crucial in the facilitation of movement. GABA is the primary inhibitory neurotransmitter in the nervous system. GABA can decrease the likelihood that action potentials will occur, and thus decreases neuronal signaling. Although it is not clear what causes neurodegeneration in PD, when a significant number of neurons have died an individual will likely start to experience movement-related problems like tremors, rigidity, slowness of movement, and postural instability.

While there is no cure for PD, treatment options vary and include medications and surgery. Surgical treatment typically involves the implantation of electrodes deep in the brain that interrupt errant signals from specific groups of neurons associated with Parkinson's symptoms with electrical impulses. Deep brain stimulation is not without risks and can sometimes result in infection or a worsening of the condition. Misalignment of the electrodes sometimes occurs and can result in sufficient damage to the integrity of the brain tissue so as to make relocation of the electrodes impossible. It is worth noting that the varying skills among surgeons can have a significant impact on surgical outcomes.

While the cause of PD is unknown, rehabilitation exercise programs with physiotherapeutic supervision have been shown to slow the progression of the symptoms while “self-help” or “self-supervised” physiotherapeutic programs have not shown similar efficacy. If a physical therapy professional is not qualified to treat PD, ineffective traditional exercises will be suggested and implemented. However, a physical therapy professional that has been educated in the treatment of PD will tailor custom activities based upon on the ambulatory abilities of the patient. Useful therapies may include a tread mill for those with sufficient motor skills, simple “weight-shifting” activities (e.g. reaching outside your base of support), rotational movements, and combining auditory vocalization with the aforementioned activities. Most of these activities are identified with one of several protocols for PD known as LSVT BIG® or variants thereof and alternative protocols. LSVT BIG® is the application of the principles of LSVT LOUD® applied to limb movement in people with PD. Specifically, LSVT BIG® training increased the amplitude of limb and body movement in people with PD is associated with improved limb movement speed, balance, and quality of life. LSVT LOUD® is an effective speech treatment for individuals with PD and other neurological conditions.

After a patient is initially diagnosed with a neurodegenerative disorder, they are typically referred to physical therapy where an initial assessment is performed. After the assessment, an initial regimen of physical therapy is prescribed and instructions are provided for them to continue these exercises unsupervised at home and return for scheduled reassessment. Unfortunately, few patients can benefit from home-based regimens because their brain is subjectively telling them that they are making the correct movement when objective evidence would show otherwise and, as a result, their progress is curtailed at best because the therapist is limited to periodic encouragement and reassessment during office visits.

In almost all cases, not just neurologically-defined therapies, therapy outside the office does not occur. Specific to neurologic disorders, such as PD, such therapies may be absolutely essential to decelerate and potentially stop further progression of symptoms. It is estimated that the typical patient diagnosed with PD will not walk after eight years of onset and will be entirely bed ridden after ten years.

### SUMMARY

The present application describes a further evolution of physiotherapeutic treatment for neurodegenerative disorders utilized in combination with other additional therapeutic techniques, devices, and methods so as to provide a therapeutic system capable of being effectively performed with the patient in their own home or in a therapy center with remote human supervision or with artificial intelligence supervision. The artificial intelligence built into the system permits the therapy to be modified as needed based upon historical or real-time feedback. Another advantage of the system is that is permits the collection of empirical data to provide a more objective evaluation of the disease progression.

After a patient is diagnosed with a neurodegenerative disorder, the patient generally does an initial regimen of physical and mental evaluations to assess the current progression of symptoms for the purpose of establishing a baseline. Based upon the initial assessment and an evaluation of patient capabilities, a customized therapeutic program is developed for the patient which targets improvements in specific physical and mental symptoms. The described method and system utilizes wearable sensors to ascertain position of various body parts and determines position over time to ascertain the control, timing, and speed of the movement so as to provide empirical data as an objective measure of symptom progression. When coupled with cognitive tasks that require a patient to accomplish a cognitive task in combination with movement, the progression of the disease can be measured and tracked over time and real-time adjustments to therapy regimens can be made to not only ascertain the disease progress should anomalous movements or decisions be detected, but also to ensure that the patient completes a requisite amount of therapy in a safe manner.

The system is comprised of a computer system arranged to receive communication from a variety of sensors that will, at the very least, measure and communicate movement of specific parts of the patient's body and mass distribution at the patient's feet. Additional sensors are incorporated into a touchpad/touchscreen to record patient touches to specific images or items. A further sensor measures and communicates eye movement. Still further sensors can capture data associated with kicking, punching, stepping/gait, and gripping in addition other motor skills and sensory inputs.

### BRIEF DESCRIPTION OF THE DRAWINGS

FIG. 1 depicts an embodiment of the configuration of system with sensors.

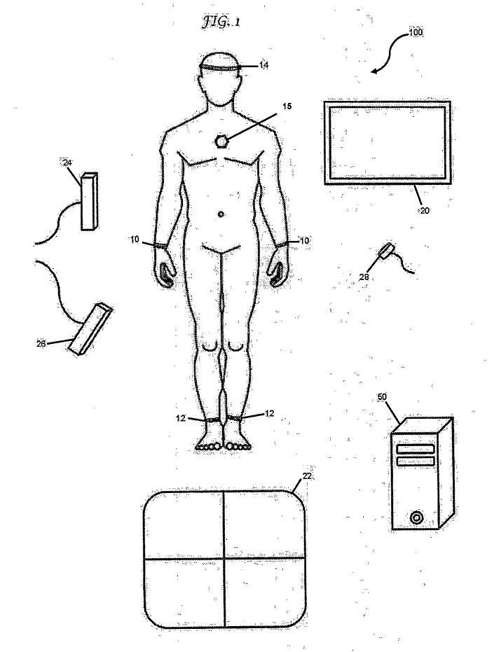

FIG. 2 depicts the process flow for dynamic regimen modification.

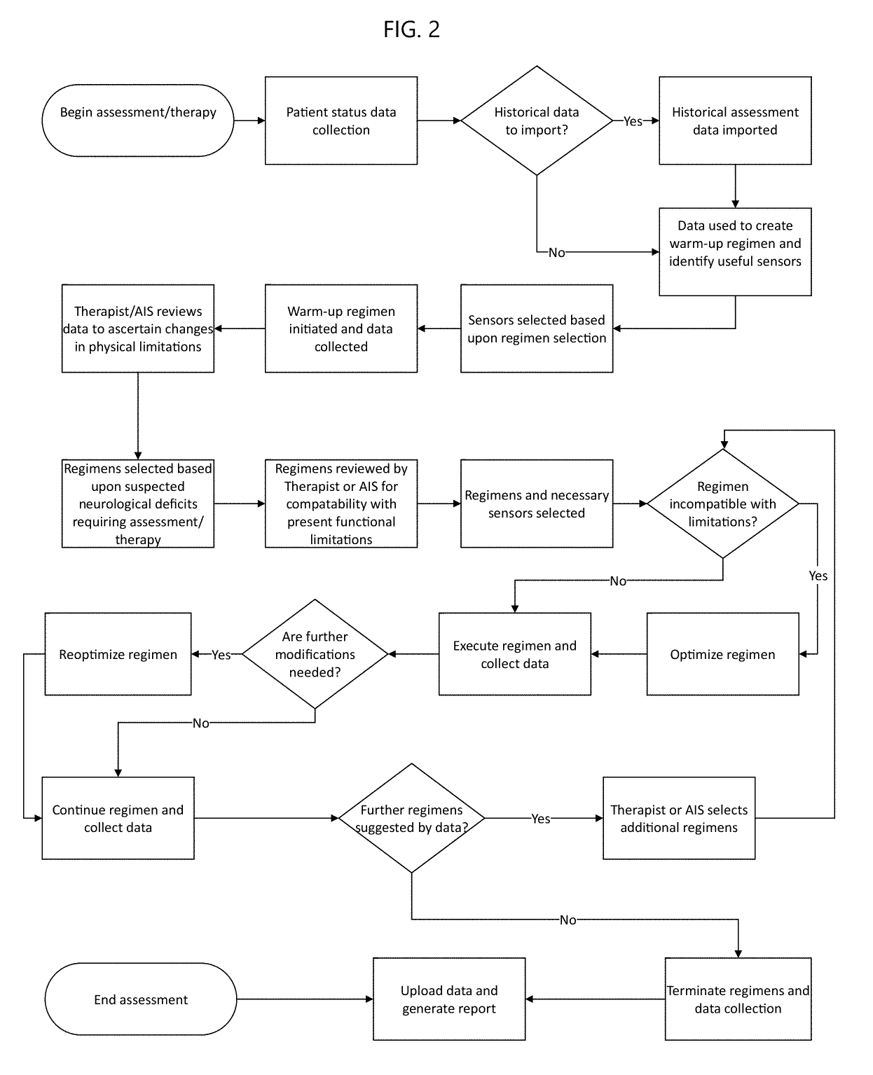

### DETAILED DESCRIPTION OF THE EMBODIMENTS

The present application describes various embodiments of a neurologic deficit assessment and therapy system 100, the System 100, for the assessment of motor skills and/or cognitive skills of patients having neurologic function deficits such as brain injuries and neurodegenerative disorders, methods of use in assessment and therapy, and a process by which the collection of sensor data permits an artificial intelligence system 200, i.e. AIS 200, installed on a computer 50 to conduct and modify physical and/or cognitive regimens administered by the System 100 based upon historical and real-time sensor data. System, as used herein, refers the physical and digital components that provide the functional aspects that provide neurologic function assessment and therapy. These components can include, but not limited to, data processing components and communication components, as well as wearable sensors, engageable sensing devices, projectors, and displays.

The following detailed description of the embodiments is intended to provide non-limiting examples of various useful embodiments utilizing the System 100, methods of use, and processes by which the System 100 can objectively measure symptoms of a neurodegenerative disorder, evaluate changes in symptomology, and modify the therapy for the purposes of assessment, improved therapeutic outcomes, and safety. Variations of these embodiments are to be expected which incorporate various sensors described herein, other sensors presently known in the art, and those sensors and systems yet to be developed which would be obvious to incorporate by one skilled in the art. The System 100 analyzes response to stimuli as interpreted through motion. The stimuli can include visual, auditory, olfactory, touch, and taste. Responses can also be chosen based on the use of cognitive skills and recorded as verbal and/or physical responses. Additionally, the patient's verbalization of words and relative loudness as well as eye movements can provide feedback for analysis.

The System 100 of the present application utilizes a plurality of sensors to obtain and communicate patient movement and cognitive skills data in the form of voluntary movements made on demand or in response to cognitive queries. The System 100 is also useful in that the wearable sensors can be worn for an extended period of time to sense involuntary movements that can provide useful information but which occur infrequently and/or randomly. The data from the sensors are treated as independent variables that are collected and processed by the System's 100 data processor for use as raw or transformed data. The data is recorded and is available as baseline historical assessment data and current assessment data. Deviations from the baseline provide insight on neurologic deficit progression over time as well as the patient's current condition and capabilities, permitting a more accurate prognosis and providing forecasting and calendaring of future needs and symptoms.

Various embodiments will utilize different configurations and types of sensors to obtain desired data and/or produce desired outcomes or diagnostics. Useful position sensors include, but are not limited to, the wrist sensors 10, ankle sensors 12, lower back sensors 14, chest sensor 15, and head sensor 14. Wearable sensors are utilized to sense gross and/or fine motor skills and report the time, rate, relative direction, and quality of movement. The delay in beginning a voluntary movement made in response to a command or made to indicate a response to a cognitive query can provide insight into neurologic deficits as can the rate of movement, the relative direction, and the quality of movement, e.g. movement continuity, completion, restrictions, and force.

An example of a commercially available wearable sensor that is useful in measuring movement includes one having 9 degrees of freedom, e.g. having a 3-axis accelerometer, a 3-axis gyroscope and a 3-axis magnetometer such as the LSM9DS1 from STMicroelectronics. The device is integrated into wearable movement/position sensor and synchronized/calibrated to provide linear acceleration, angular rate and magnetic field data which can be utilized to provide complete position and movement data.

In an embodiment, wearable sensors are utilized that can receive position related information from a neighboring wearable sensor using a magnetic near field communication (NFC) unit. For example, the position related information may include information associated with a distance, e.g. signal strength, from other wearable devices as estimated using a signal from the other wearable devices and the relative or known position of neighboring wearable devices.

In further useful embodiments, additional sensors can provide useful data about heart rate, body temperature, blood oxygenation, heart rhythm, cerebral blood flow, blood glucose, blood pressure, cerebral activity, eye movement, respiration rate, and respiratory volume. In a still further embodiment, a microphone is utilized to detect sounds and verbalizations for assessment and therapy for neurological deficits related to hearing and speech. White noise may be communicated to the patient during verbalization regimens to retrain the patient to speak at an appropriate volume. In other useful embodiments, cerebral activity and cerebral blood flow can be evaluated using functional neuroimaging during neurological deficit assessment and/or therapy.

The footpad 22 and touchpad 20, as non-limiting examples, can be pressure sensitive LCD displays that can withstand the pressure exerted on them but also register the pressure as the selection of the displayed information, e.g. an image, object, shape, pattern, number, word, letter, or color. Alternatively, the displayed information can be projected onto the surface.

A pressure sensitive kickpad 26, in a non-limiting example, could be stationed just above the knee to register the force with which the patient can raise their leg. Alternatively, or in combination with the foregoing, a pressure sensitive kickpad 26 could be stationed near the foot to register the force with which a patient can move their foot forward.

A pressure sensitive punchpad 24, in a non-limiting example, could be stationed in front of the patient to register the force with which the patient can strike forward with their hand, fist, or arm. A pressure sensitive pinchpad can be utilized to measure the pressure that can be exerted between fingers in a pinching action. Alternatively, the pinchpad can measure how well the fingers line up upon pinching to assess fine motor skills. A pressure sensitive dial sensor 28 may be incorporated as a further assessment of fine motor skills of the hand and to measure the torque that can be generated by the fingers and wrist in a rotational motion. A similar pressure sensitive grip sensor can be incorporated to measure the gripping force of the patient. A pressure sensitive lever or joystick sensor device can similarly be utilized to measure the available force in causing the movement of a lever or joystick and to assess hand control through the movement of the lever or joystick. All of the foregoing sensors can be utilized in various regimens for therapeutic as well as assessment purposes. Still further available sensors monitor eye movement for both voluntary and involuntary movement.

Additional sensors to capture responses based upon brain function related to all types of sensory input can be incorporated and used in regimens created to assess and provide cognitive and physical therapy for patients. A patient's sense of smell can be utilized as a basis for eliciting response indicated by a motor skill as can touch, temperature association (i.e. hot versus cold, or degrees of hot and cold), sound, memory, emotion recognition, and any other input that can be sensed by the patient. The primary goal is to have the patient perform the cognitive skill and respond with a motor skill so that the assessment and therapy produce objective evidence to qualify and quantify neurodegenerative disorder symptoms to minimize the reliance upon subjective determinations and to provide more effective assessments and therapies based upon current and historical data.

The types of devices and sensors to measure and record movement and force, in addition to testing other sensory inputs such as smell and touch, that would be useful in practicing the methods disclosed herein are far too numerous to list in this application but would be apparent to one skilled in the art upon reading this specification and claims and understanding the objectives of the System and method disclosed herein. Presently known sensors that are capable of being adapted to record physical movement or decisions are incorporated by reference as if fully described herein.

An artificial intelligence system 200, AIS 200, is coupled with the System to provide optimized regimens to the patient. These physical and/or cognitive regimens may be utilized to assess neurological, physiological, and physical deficits in a patient using data collected from various sensors and through the assessment of requested voluntary motor skills and responsive voluntary motor skills. These physical and/or cognitive regimens may be utilized to provide therapy to affect the progression of symptoms associated with a patient's neurological deficits and/or physical deficits. A review of historical assessment data by the AIS 200 may result in a modification of prescribed regimens in an effort to better assess the patient's condition, improve therapeutic results, or to improve safety.

Appropriate regimens can be modeled after data sets to achieve optimal results. Neural networks may also be utilized to better assess the data and provide more effective modifications of regimens as well as predictions of motor skill failures such as an impending fall or the future loss of mobility.

The System and associated methods of use are also beneficial in providing valuable objective research data, even across generations. A broader range of symptoms and their progression across neurodegenerative disorders can be objectively evaluated by mapping cognitive and motor skill degradation over time and will lead to a better understanding of such disorders, their origins, the affected areas of the brain, and the evaluation and development of potential therapies and cures in ways that were previously not possible.

### Example 1

A patient has been assessed by the AIS and the data suggests that their Parkinson's Disease, i.e. PD, may have progressed from stage 2 to stage 3 due to a reported slowing of movement. Slowness of movement is often reported with stage 2 PD but is also associated with advancing age. Progression from stage 2 to stage 3 of PD is often accompanied by an increased slowing of movement and may be indicative of the progression of the disease. The AID, upon detecting the increasing slowness of movement, then includes additional regimens intended to better assess the presence of stage 3 PD symptoms. Regimens for balance are then included by the AIS or, if balance regimens were already included, additional balance regimens can included to more fully assess balance issues in the patient. Noting the progression of PD, the AID also incorporates additional therapeutic regimens intended to improve the patient's involuntary and voluntary responses to balance adjustment.

The System also permits the self-reporting of limitations by the patient for use by the AIS 200 in selecting and/or modifying prescribed regimens. The patient may have experienced an injury that needs to be accommodated by the system, or perhaps they are experiencing temporary fluctuations in symptom severity and could benefit from targeted assessment and therapy or a reduction or increase in goals for various regimens. Warm up regimens could also incorporate assessment data to permit the AIS 200 or a remote or local therapist to adjust regimens for optimal assessment, therapeutic, and safety objectives as needed.

Some data, e.g. data from wearable sensors, can be locally stored on the patient and then uploaded. Alternatively, the data can be communicated in real time for storage to a nearby digital storage media or for immediate upload to the System. The System may operate offline from any remote, e.g. cloud, based systems. Patient data could subsequently be uploaded over the internet or retrieved locally via transfer of digital storage media to a therapist. Alternatively, the System may operate on-line and upload the data to a remote system in real-time or lag-time. The AIS 200 may likewise be arranged locally with the System at the patient's residence or a local provider, or alternatively may be arranged remotely, e.g. cloud-based. Interaction with and review by a therapist could likewise take place in real-time, or the therapist could prescribe and schedule appropriate regimens based upon a review of available data.

### Example 2

A patient is experiencing physical and cognitive impairment from PD and is exhibiting symptoms that indicate a deficit in problem solving skills in combination with a marked change in gait. The AIS introduces regimens intended to improve gait and problem solving by using stepping motions to target areas of a pressure sensitive footpad that indicate possible responses to the problems communicated by the System. In this example, the target areas of the footpad are identified by numbers displayed on the footpad and the patient may be asked to solve a simple math problem, thus incorporating cognitive skills therapy with physical therapy. An additional therapy is also included by the AIS 200 which introduces gamification into the physical therapy that requires the patient to step more quickly to designated areas and at increasing distances to succeed at the game. The AIS 200 limits the stepping distance based upon available historical data which indicates that the patient has an increased chance of falling due to gait and balance issues when they step at a distance greater than 0.5 meters. Sensing a present increase in loss of balance at 0.5 meters during the therapy, the AIS 200 further restricts the stepping distance during the therapy to accommodate current physical restrictions and to improve patient safety while still completing, and benefiting, from a therapeutic session.

What is claimed is:

1. A method for the assessment of neurologic function for the purpose of at least one of medical evaluation and research, comprising:
a. assessing neuromuscular responses by causing a person to perform at least one of a voluntary motor skill or sequence of voluntary motor skills while using at least one sensor to collect objective motion related data made in response to said voluntary motor skill; and
b. assessing cognitive skills by causing said person to indicate their response to a query through at least one responsive voluntary motor skill or sequence of responsive voluntary motor skills associated with at least one response to said query and gathering objective data related to said responsive voluntary motor skill or sequence of responsive voluntary motor skills.

2. The method of claim 1, wherein said requested voluntary motor skills are at least one of gross motor skills and fine motor skills and said responsive voluntary motor skills are at least one of gross motor skills and fine motor skills.

3. The method of claim 2, wherein said cognitive skills assessed includes at least one of logical reasoning, mathematical processing, memory, image recognition, name recognition, name association, color association, color recognition, word association, word recognition, linguistic skills, reading comprehension, word recognition, word association, sound recognition, sound association, smell recognition, smell association, taste recognition, and taste association.

4. The method of claim 3, further comprising the use of neuroimaging to assess cerebral activity related to at least one of requested voluntary motor skills, responsive voluntary motor skills, and cognitive skills.

5. The method of claim 2, wherein said data is utilized to perform at least one of an evaluation of present neurologic function, the creation of a baseline of neurologic function, and an evaluation of neurologic function over time.

6. The method of claim 5, wherein motion sensors are placed on body locations including at least one of a wrist, an ankle, the chest, the lower back, and the head.

7. The method of claim 6, wherein an artificial intelligence system is utilized to provide therapy for at least one of a neurologic deficit and motor skill deficit, and said data collected is utilized in at least one of a raw and mathematically transformed state to prescribe therapeutic activities based upon at least one of historical and present data.

8. The method of claim 7, wherein said artificial intelligence system is programmed to select at least one of an assessment regimen and a therapeutic regimen based upon at least one of patient capabilities and patient therapeutic needs as indicated by at least one of present assessment results and historical assessment results related to said patient's response to at least one of a requested voluntary motor skill and a responsive voluntary motor skill.

9. The method of claim 8, wherein said artificial intelligence system modifies said regimens in real-time based upon present assessment results.

10. The method of claim 8, wherein said system is utilized to provide at least one of a remote assessment of a patient and remote therapy of a patient.

11. The method of claim 10, wherein at least one of said remote assessment and said remote therapy is conducted with at least one of minimal or absent human supervision.

12. The method of claim 10, wherein said system communicates data and selected regimens over an electronic communications network.

13. A method for the rehabilitation of patients having a neurologic function deficit for at least one of the purposes of medical treatment and research, comprising:
a. producing neuromuscular responses in a patient by causing said patient to perform at least one of a voluntary motor skill or sequence of voluntary motor skills while using at least one sensor to collect objective motion related data made in response to said voluntary motor skill; and
b. producing cognitive responses associated with voluntary motor skills by causing said patient to indicate their response to a query through at least one responsive voluntary motor skill or sequence of responsive voluntary motor skills associated with at least one response to said query and gathering objective data related to said responsive voluntary motor skill or sequence of responsive voluntary motor skills.

14. The method of claim 13, wherein said voluntary motor skills are at least one of gross motor skills and fine motor skills and said responsive voluntary motor skills are at least one of gross motor skills and fine motor skills.

15. The method of claim 14, wherein said cognitive skills include at least one of logical reasoning, mathematical processing, memory, image recognition, name recognition, name association, color association, color recognition, word association, word recognition, linguistic skills, reading comprehension, word recognition, word association, sound recognition, sound association, smell recognition, smell association, taste recognition, and taste association.

16. The method of claim 15, wherein said data is utilized in at least one of a raw and mathematically transformed state to prescribe therapeutic activities based upon historical and/or present observations.

17. The method of claim 16, wherein an artificial intelligence system is utilized to provide therapy for at least one of a neurologic deficit and motor skill deficit, and said data collected is utilized in at least one of a raw and mathematically transformed state to prescribe therapeutic activities based upon at least one of historical and present data.

18. The method of claim 17, wherein said artificial intelligence system is programmed to select at least one of an assessment regimen and a therapeutic regimen based upon at least one of patient capabilities and patient therapeutic needs as indicated by at least one of present assessment results and historical assessment results related to said patient's response to at least one of a requested voluntary motor skill and a responsive voluntary motor skill.

19. The method of claim 18, wherein said artificial intelligence system modifies said regimens in real-time based upon present assessment results.

20. The method of claim 18, wherein said system is utilized to provide at least one of a remote assessment of a patient and remote therapy of a patient.

21. The method of claim 20, wherein at least one of said remote assessment and said remote therapy is conducted without human supervision.

22. The method of claim 20, wherein said system communicates data and selected regimens over an electronic communications network.

23. A system for the assessment of neurologic function comprising sensors that can report three-dimensional movement.

24. The system of claim 23, wherein at least one of said sensors possesses a gyroscope and at least one of an accelerometer and a magnetometer.

25. The system of claim 24, further comprising sensors that report one or more of heart rate, body temperature, blood oxygenation, heart rhythm, cerebral blood flow, blood glucose, blood pressure, cerebral activity, eye movement, respiration rate, and respiratory volume.

26. The system of claim 25, wherein said sensors wirelessly communicate data to said system.

27. The system of claim 25, further comprising at least one of a microphone for transmitting sound made by or verbalized by a patient, a touchpad that senses touch location, a pressure sending kickpad, a pressure sensing punchpad, a dial sensor device that senses at least one of movement and force, a lever sensor device that senses at least one of movement and force, a pressure sensing finger pinchpad, and a footpad that senses at least one of pressure and pressure location.

28. The system of claim 27, wherein said touchpad bears at least one object or image to be touched and senses a touch made in response to a physical objective of at least one of an assessment or therapeutic regimen.

29. The system of claim 27, further comprising finger worn sensors.

30. The system of claim 23, wherein said system is programmed to communicate a regimen of at least one of physical activities and cognitive activities to a patient, is capable of receiving and processing sensor data from said sensors, is capable of reporting results over time, and is capable of selectively modifying said communicated regimen in response to at least one of historical assessment data and current assessment data and input from a therapist so as to optimize at least one of neurologic function assessment and neurologic function therapy.

31. The system of claim 24, wherein sensor data is stored locally and electronically communicated to the system for processing.
